(19) United States
(12) Patent Application Publication
(10) Pub. No.: US 2016/0000787 A1
(43) Pub. Date: Jan. 7, 2016

(54) INHIBITORS OF CDK8/19 FOR USE IN TREATING ESTROGEN RECEPTOR POSITIVE BREAST CANCER

(71) Applicant: SENEX BIOTECHNOLOGY, INC., Columbia, SC (US)

(72) Inventors: Eugenia Broude, Lexington, SC (US); Igor B Roninson, Lexington, SC (US)

(73) Assignee: SENEX BIOTECHNOLOGY, INC., Columbia, SC (US)

(21) Appl. No.: 14/770,296

(22) PCT Filed: Feb. 26, 2014
(86) PCT No.: PCT/US14/18678

§ 371 Date: Aug. 25, 2015

Related U.S. Application Data
(60) Provisional application No. 61/769,435, filed on Feb. 26, 2013.

(51) Int. Cl. A61K 31/517; A61K 31/565; A61K 31/5377; A61K 45/06

(52) U.S. Cl. CPC A61K 31/517; A61K 45/06; A61K 31/565; A61K 31/5377

(57) ABSTRACT

The invention provides a selective inhibitor of CDK8/19 for use in a method of treating a patient having estrogen receptor positive (ER+) breast cancer, including breast cancer that is resistant to antiestrogen therapy. In some embodiments, the selective inhibitor of CDK8/19 is administered in combination with antiestrogen therapy. In some embodiments, the selective inhibitor of CDK8/19 is administered to ER+HER2+ breast cancer patients in combination with HER2-targeting drugs.

### BACKGROUND OF THE INVENTION

1. Field of the Invention

The invention relates to the treatment of estrogen receptor positive (ER+) breast cancer.

2. Summary of the Related Art

The ACS estimates that about 232,340 new cases of invasive breast cancer and about 64,640 new cases of carcinoma in situ will be diagnosed in women in 2013 in the US, and about 39,620 women will die from breast cancer. At least 75% of breast cancers express estrogen receptor (ER), a steroid hormone receptor that regulates transcription, and/or progesterone receptor (PR), biomarkers of estrogen dependence. Such patients usually receive adjuvant antiestrogen therapy, following surgery. Antiestrogen drugs may inhibit ER by antagonizing estrogen ligand binding to ER or modulating ER activity (tamoxifen and other selective ER modulators, SERMs), inhibiting dimerization and downregulating ER (fulvestrant and other selective estrogen receptor downregulators, SERDs), or blocking estrogen production (aromatase inhibitors, AIs) (Sweeney et al., 2012; McDonnell and Wardell, 2010). Premenopausal women with ER+BrCa are usually prescribed adjuvant therapy combining tamoxifen with cytotoxic chemotherapy, while post-menopausal women with ER+BrCa are likely to receive an AI. If these treatments fail to prevent relapse, fulvestrant is used as a second line antiestrogen therapy (Catania et al., 2007). Unfortunately, many tumors exhibit either de novo or acquired resistance to antiestrogen treatments. The mechanisms of such resistance are varied and include changes in ER, such as the appearance of hypersensitive ER stimulated by very low doses of estrogen, the emergence of ER with ligand-independent activity, and the downregulation or loss of ER coupled with the activation of various signaling mechanisms that activate ER-regulated genes required for cell proliferation; the latter category includes, among others, PI3K, HER2/NEU and EGFR (Sweeney et al., 2012). Therapeutic approaches targeting factors that potentiate the transcriptional effects of ER are actively exploited to increase the efficacy of the antiestrogen therapy, with much of the preclinical and clinical research concentrating on inhibitors of receptor tyrosine kinases (RTKs), such as HER2/NEU or EGFR (Sweeney et al., 2012). However, the efficacy of the RTK inhibitors is invariably limited by the emergence of drug resistance, due primarily to the fact that increased levels of RTK ligands render tumor cells resistant to these drugs (Wilson et al., 2012). Identification of new “druggable” mediators of the ER-regulated mitogenic effects could help in developing new approaches to the treatment of antiestrogen-resistant cancers.

CDK8 (ubiquitously expressed), along with its closely related isoform CDK19 (which is expressed in only a subset of tissues), is an oncogenic transcription-regulating kinase (Xu and Ji, 2011; Galbraith et al., 2010; Firestein and Hahn, 2009). In contrast to better-known members of the CDK family (such as CDK1, CDK2, and CDK4/6), CDK8 plays no role in cell cycle progression. CDK8 knockout in embryonic stem cells prevents embryonic development (Westerling et al., 2007), due to its essential role in the pluripotent stem cell phenotype (Adler et al., 2012) but CDK8 depletion does not inhibit the growth of normal cells (Westerling et al., 2007; Firestein et al., 2008). Furthermore, CDK8 inhibitors are neither cytotoxic nor cytostatic to normal cells or to most of the tested tumor cell types (Porter et al., 2012), which distinguishes them from almost all of the approved and experimental cancer agents. Instead, the role of CDK8 in cancer is due to its unique function as a regulator of several transcriptional programs involved in carcinogenesis (Xu and Ji, 2011) and chemotherapeutic drug response (Porter et al., 2012). CDK8 has been identified as an oncogene in melanoma (Kapoor et al., 2010) and colon cancer (Firestein et al., 2008), the CDK8 gene being amplified in ˜50% of the latter cancers. While higher expression of CDK8 has been associated with worse prognosis in colon cancer (Firestein et al., 2010), the strongest prognostic correlations for CDK8 expression have been found so far in a bioinformatics analysis of microarray data from 2,897 breast cancer patients, where above-median expression of CDK8 was associated with 7-8 years shorter relapse-free survival (RFS) (Porter et al., 2012). Since the majority of these patients were ER+ and were likely to receive endocrine therapy, this raised a possibility that CDK8 could be involved in ER signaling in breast cancers. In particular, CDK8 could act as a positive effector of ER (as it does for the thyroid hormone receptor (Belakavadi and Fondell, 2010)), thereby enabling tumor cells with low ER to utilize the mitogenic estrogen signal more efficiently. In this case, CDK8 inhibition could inhibit estrogen-dependent breast cancer cell growth and sensitize ER+ breast cancers to endocrine therapy.

### BRIEF SUMMARY OF THE INVENTION

The invention provides methods for treating a patient having estrogen receptor positive (ER+) breast cancer. The methods according to the invention comprise administering to the patient an effective amount of a selective inhibitor of CDK8/19. In some embodiments, the breast cancer to be treated is resistant to antiestrogen therapy. In many cases of such resistant cancers, the cancer cells express one or more of the genes GREB1, CXCL12, and TFF.

In some embodiments, the selective inhibitor of CDK8/19 is administered in combination with treating the patient with antiestrogen therapy. In some embodiments, the antiestrogen therapy comprises administering to the patient a selective estrogen receptor modulator, a selective estrogen receptor downregulator, or an aromatase inhibitor. In some embodiments a selective inhibitor of CDK8/19 is used in combination with a HER2 inhibitor to treat ER+, HER2+ breast cancers.

### BRIEF DESCRIPTION OF THE DRAWINGS

FIG. 1 (A, B, C) shows examples of selective inhibitors of CDK8/19 that are useful in the methods according to the invention.

FIG. 2 shows a synthesis scheme for Senexin B and similar compounds.

Scheme 1. a. NaBH₄, THF, MeOH, 60° C., 6 h; b. CBr₄, Ph₃P, DCM, r.t., 12 h; c. NaCN, CH₃CN/HO₂O, reflux, 12 h; d. Boc₂O, NaBH₄, NiCl₂.6H₂O, MeOH, r.t., 12 h; e. NaOH, THF/H₂O, r.t., 4 h; f. 1-methyl-piperazine, NEt₃, TBTU, DCM, r.t., 6 h; g. HCl/dioxane, THF, r.t., 3 h; h. Mel, K₃CO₃, DMF, r.t., 12 h; i. formamide, reflux, 2-3 h; j. SOCl₂, reflux, 2 h k. [6-(2-Amino-ethyl)-naphthalen-2-yl]-(4-methyl-piperazin-1-yl)-methanone dihydrochloride (8), NEt₃, CH₃CN, reflux, 1 h; l. Zn(CN)₂, (1,1′-bis(diphenyl-phosphino)ferocene)-dichloro-palladium (II), N,N-dimethylacetamide, 120° C., 2 h

FIG. 3 shows effects of selective CDK8/19 inhibitor Senexin A on estrogen-stimulated cell proliferation of MCF7 cells.

FIG. 4 shows effects of different concentrations of selective CDK8/19 inhibitors Senexin A and Senexin B on the growth of ER+ breast cancer cell lines MCF7 (FIG. 4A), T47D (FIG. 4B), and BT474 (FIG. 4C).

FIG. 5 shows effects of Senexin A on growth inhibition by ER inhibitor fulvestrant in completely or partially estrogen-independent cell lines MCF7-1p and MCF7-Veh (FIG. 5a) and on estrogen-stimulated MCF7 cell growth (FIG. 5B).

FIG. 6 shows effects of Senexin A alone and in combination with HER2 and EGFR inhibitor lapatinib (FIG. 6A,B) and with a humanized monoclonal antibody against HER2 (FIG. 6C) on ER+HER2+BT474 cells.

FIG. 7 shows results of QPCR analysis of the effects of E2 and Senexin A on the expression of estrogen-inducible genes in MCF7 cells.

FIG. 8 (A, B) shows the effect of Senexin A on ER-dependent promoter activity in T47D-ER/Luc cells.

### DETAILED DESCRIPTION OF THE PREFERRED EMBODIMENTS

The invention provides methods for treating a patient having estrogen receptor positive (ER+) breast cancer. The methods according to the invention comprise administering to the patient an effective amount of a selective inhibitor of CDK8/19. In some embodiments, the breast cancer to be treated is resistant to antiestrogen therapy. In many cases of such resistant cancers, the cancer cells express one or more of the genes GREB1, CXCL12, and TFF.

In some embodiments, the selective inhibitor of CDK8/19 is administered in combination with treating the patient with antiestrogen therapy. In some embodiments, the antiestrogen therapy comprises administering to the patient a selective estrogen receptor modulator, a selective estrogen receptor downregulator, or an aromatase inhibitor. In some embodiments, the selective estrogen receptor modulator is tamoxifen, raloxifine, or toremifine. In some embodiments, the selective estrogen receptor downregulator is fulvestrant. In some embodiments, the aromatase inhibitor is anastrozole, exemestane, or letrozole. In some embodiments a selective inhibitor of CDK8/19 is used in combination with a HER2 inhibitor, non-limiting examples are trastuzumab or lapatinib, to treat ER+, HER2+ breast cancers.

Selective inhibitors of CDK8/19 useful in the methods according to the invention have been described in U.S. patent application Ser. No. 13/757,682.

In some embodiments, the selective inhibitor of CDK8/19 has structural formula I or II:

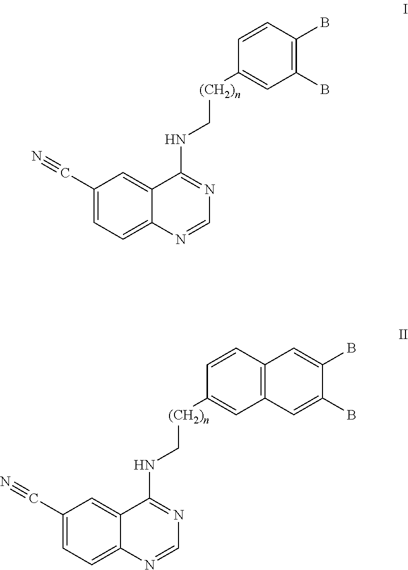

wherein each B is independently hydrogen or

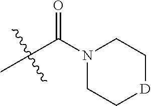

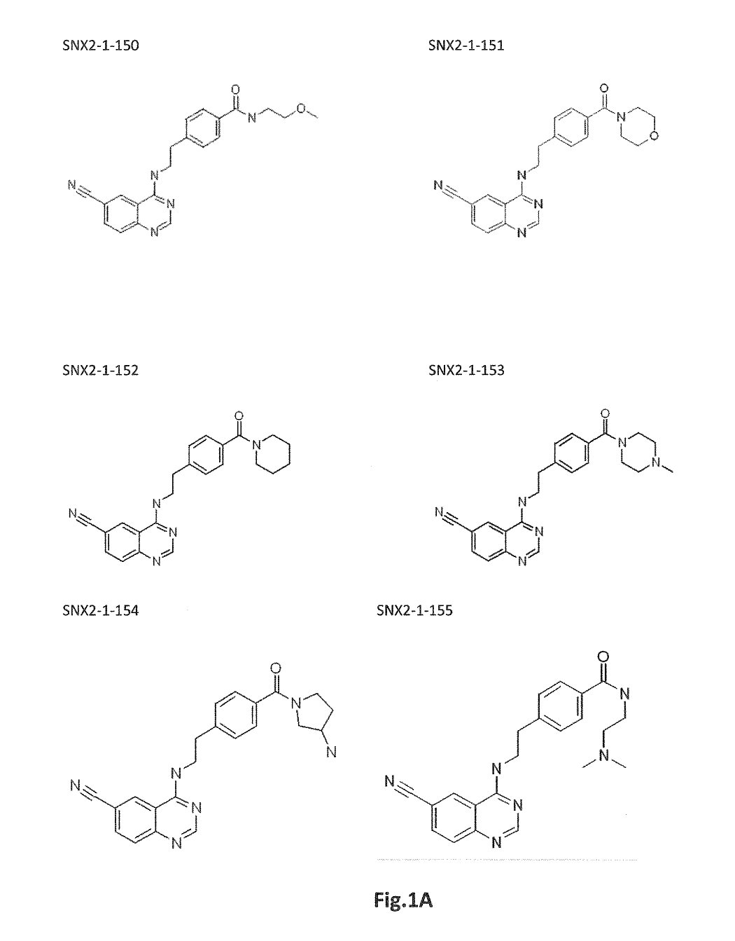

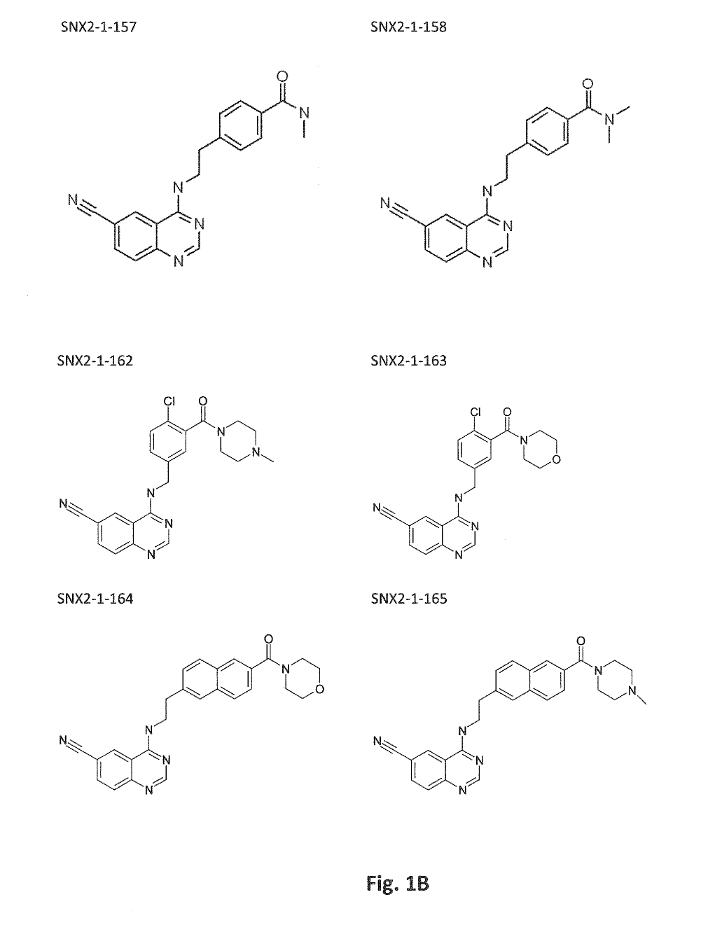

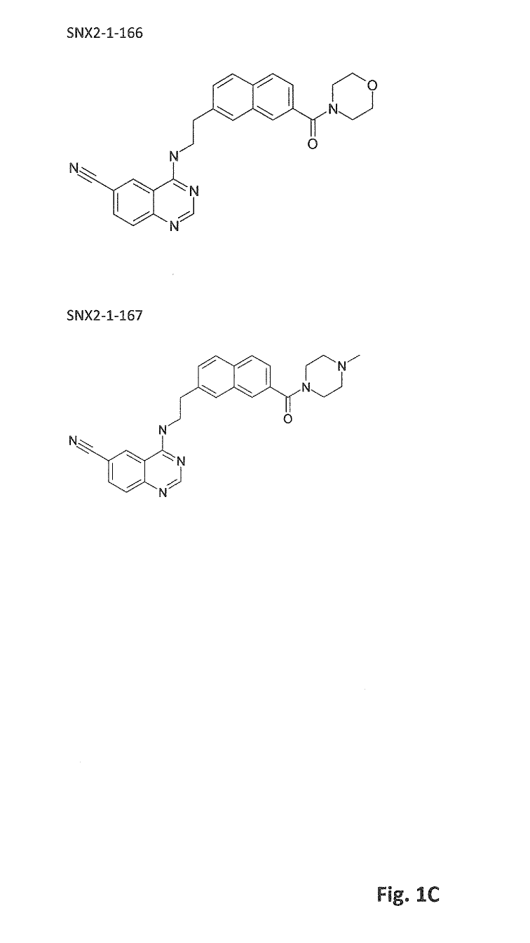

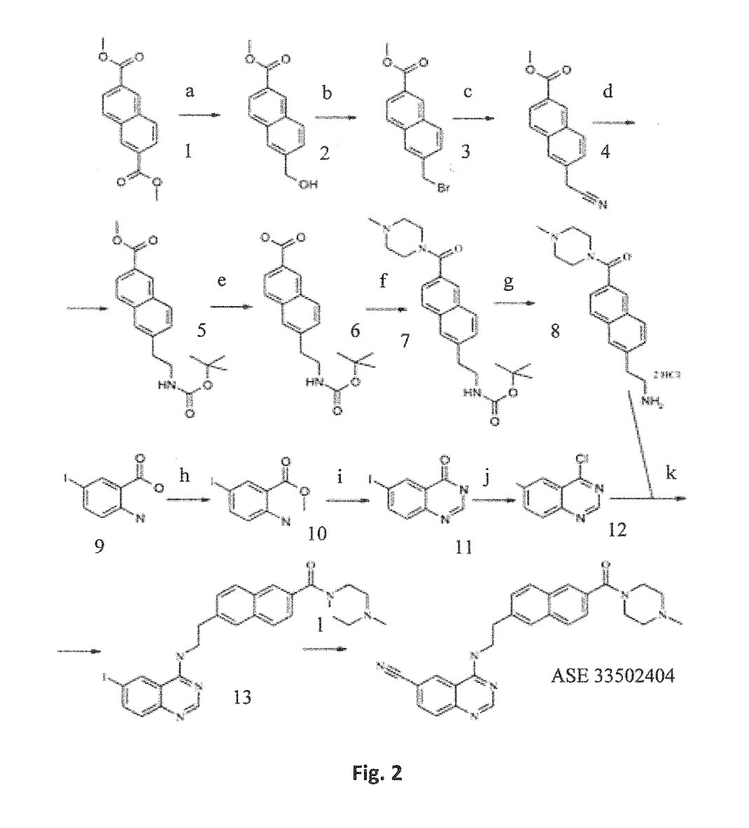

provided that at least one B is hydrogen and not more than one B is hydrogen;
D is selected from —NH, —N-lower alkyl, or O; and n is 0-2. “Lower alkyl” means an alkyl radical of 1-6 carbon atoms, which may be linear or branched. In some embodiments, the lower alkyl is methyl and n is 0 or 1. In some embodiments, the selective inhibitor of CDK8/19 is SNX2-1-162, SNX2-1-163, SNX2-1-164, SNX2-1-165, SNX2-1-166, or SNX2-1-167. In some embodiments the selective inhibitor of CDK8/19 is SNX2-1-165. In some embodiments, the selective inhibitor of CDK8/19 is selected from the compounds shown in FIG. 1. In some embodiments, the selective inhibitor of CDK8/19 is administered orally.

For purposes of the invention a selective inhibitor of CDK8/19 is a small molecule compound that inhibits one or more of CDK8 and CDK19 to a greater extent than it inhibits certain other CDKs. In some embodiments, such compounds further inhibit CDK8/19 to a greater extent than CDK9. In preferred embodiments, such greater extent is at least 2-fold more than CDK9. A “small molecule compound” is a molecule having a formula weight of about 800 Daltons or less. The term “in combination with” means that two different agents may be administered in any order, including simultaneous administration, as well as temporally spaced order from a few seconds up to several days apart. Such combination treatment may also include more than a single administration of the selective inhibitor of CDK8/19 and/or independently the antiestrogen therapeutic

In the methods according to the invention, the compounds described above may be incorporated into a pharmaceutical formulation. Such formulations comprise the compound, which may be in the form of a free acid, salt or prodrug, in a pharmaceutically acceptable diluent (including, without limitation, water), carrier, or excipient. Such formulations are well known in the art and are described, e.g., in Remington's Pharmaceutical Sciences, 18th Edition, ed. A. Gennaro, Mack Publishing Co., Easton, Pa., 1990. The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. As used herein, the term “pharmaceutically acceptable salts” refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate). The active compound is included in the pharmaceutically acceptable carrier or diluent in an amount sufficient to deliver to a patient a therapeutically effective amount without causing serious toxic effects in the patient treated. A “therapeutically effective amount” is an amount sufficient to alleviate or eliminate signs or symptoms of the disease. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art. In certain applications, an effective dose range for a 70 kg patient is from about 50 mg per patient per day up to about 10 grams per patient per day, or the maximum tolerated dose. In certain preferred embodiments the dose range is from about 200 mg per patient per day to about 10 g per patient per day. In certain preferred embodiments the dose range is from about 200 mg per patient per day to about 5 g per patient per day. The dose in each patient may be adjusted depending on the clinical response to the administration of a particular drug. Administration of the pharmaceutical formulations in the methods according to the invention may be by any medically accepted route, including, without limitation, parenteral, oral, sublingual, transdermal, topical, intranasal, intratracheal, or intrarectal. In certain preferred embodiments, compositions of the invention are administered parenterally, e.g., intravenously in a hospital setting. In certain other preferred embodiments, administration may preferably be by the oral route.

The following examples are intended to further illustrate certain preferred embodiments of the invention and are not intended to limit the scope of the invention.

### Example 1

### CDK8/19 Inhibition Inhibits Mitogenic Effect of Estrogen in ER+ Breast Cancer Cells

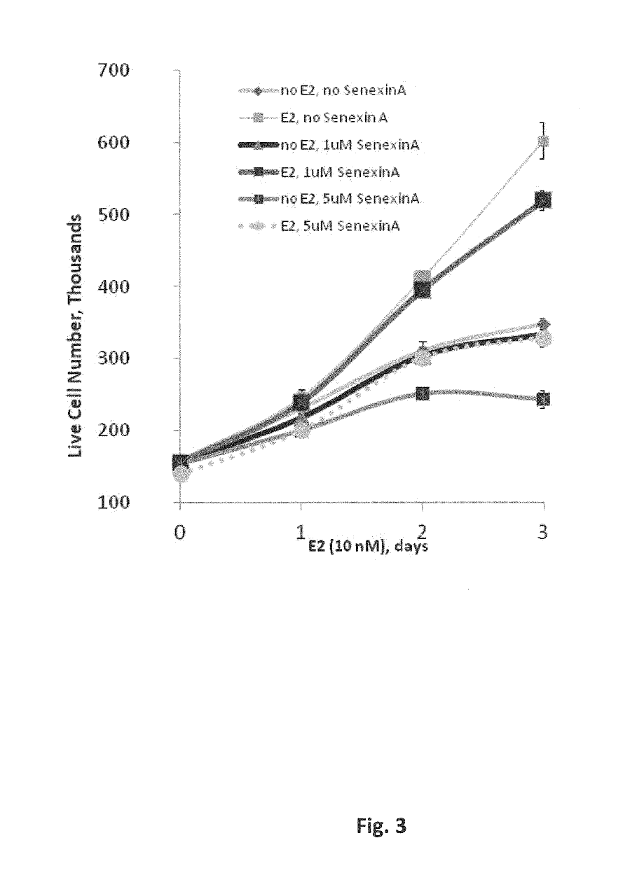

To test the effect of CDK8/19 inhibition on the mitogenic effect of estrogen in estrogen-responsive BrCa cells, MCF7 cells (ER+BrCa line) were placed into estrogen-depleted phenol red-free media with 10% charcoal-stripped serum. Under these conditions cells are largely but not completely estrogen-depleted (full depletion would require adapting the cells to low serum, since the cells can synthesize estrogen from serum components). Cells were then plated in the presence or absence of Senexin A (at 1 μM and 5 μM concentrations), and either 10 nM of the estrogen 17-β-estradiol (E2) or vehicle control were added on the following day. Cell growth was measured by flow cytometric counting of live (PI-negative) and dead (PI-positive) cells over 4 days. As shown in FIG. 3, E2 strongly stimulated cell growth, but this effect was abolished with 5 μM Senexin A (1 μM Senexin A produced a partial effect). 5 μM Senexin A also inhibited cell growth without E2 addition but to a lesser extent (FIG. 3; this effect may potentially be mediated by estrogen that MCF7 cells synthesize from 10% serum). The effects of Senexin A were cytostatic and were not associated with any significant induction of cell death. Hence, CDK8 inhibition abolishes estrogen stimulation in MCF7 cells, suggesting that CDK8 may be an effector of ER-mediated mitogenic activity.

### Example 2

### CDK8/19 Inhibition Inhibits the Growth of ER+ Breast Cancer Cells and Potentiates the Effects of Antiestrogen Drugs

The effect of Senexin A on mitogenic response to E2 suggested that CDK8/19 inhibitors could inhibit the growth of estrogen-dependent BrCa cells in regular (estrogen-containing) media, in contrast to the inhibitor's lack of growth inhibition in most other cell types, and that they may also potentiate the effects of antiestrogen drugs in both estrogen-dependent and estrogen-independent ER+ cell lines. Table 1 shows the growth-inhibitory effects of Senexin A alone (tested at 5 μM) and in combinations with a SERD (fulvestrant) and a SERM (tamoxifen) in MCF7 and T47D (ER+HER2−) and BT474 (ER+HER2+, fulvestrant-resistant) cell lines, and in two derivatives of MCF7, MCF7-Veh and MCF7-1pE, selected by long-term growth in estrogen-depleted media and in media supplemented with a very low (1 pM) concentration of E2, respectively (Sikora et al., 2012). Both MCF7-1pE and MCF7-Veh are resistant to tamoxifen and MCF7-Veh is also resistant to fulvestrant.

TABLE 1
Growth inhibitory effects of Senexin A, alone and in combinations
with fulvestrant (FUL) or 4-OH tamoxifen (TAM) in the indicated cell
lines. Cells were treated for 6 days, in triplicates and counted using
Coulter counter. The numbers show % growth inhibition relative to
control (mean of triplicate measurements; the standard errors did not
exceed 7% of the mean), colored on the green-yellow-red color scale.

| | T47D | MCF7 | MCF7-1pE | MCF7-Veh | BT474 |
|---|---|---|---|---|---|
| SenA (5 μM) | 48 | 37 | 19.1 | 15.5 | 23.9 |
| FUL (1 nM) | 56.5 | 62 | 49.7 | 12.4 | 17.7 |
| SenA + FUL (1) | 76.2 | 72.2 | 65.7 | 32 | 32.7 |
| FUL (10 nM) | 58.2 | 63.6 | 57.2 | 28 | 22.6 |
| SenA + FUL (10) | 71.6 | 74.8 | 72 | 43.7 | 42.4 |
| TAM (1 μM) | 54.6 | 34.8 | −1.3 | 2.6 | 28.8 |
| SenA + TAM (1) | 70.5 | 62.1 | 26.1 | 23.8 | 35.3 |
| TAM (5 μM) | 62.3 | 50.7 | 1.5 | 1 | 42.3 |
| SenA + TAM (5) | 75.7 | 68.9 | 29.9 | 23.6 | 46.9 |

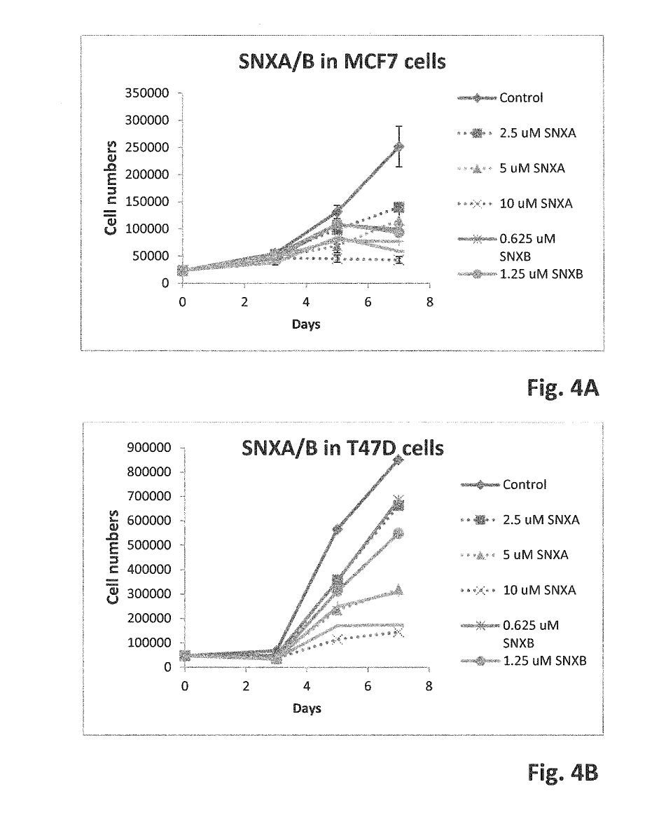

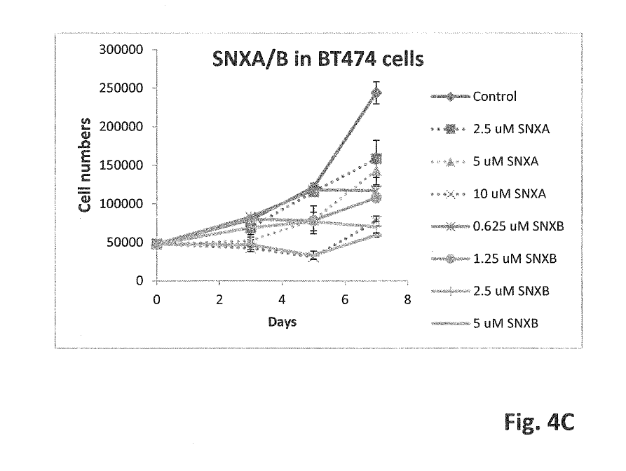

Senexin A alone inhibited cell growth, with the strongest effects observed in the estrogen-dependent lines T47D and parental MCF7 and the weakest effects in cell lines that are fully or partially estrogen-independent (MCF7-Veh, MCF7-1p). FIG. 4 shows the effects of different concentrations of CDK8/19 inhibitors Senexin A and Senexin B on time course of cell growth of MCF7, T47D and BT474 cells over 7 days, with the cell numbers measured using Coulter counter. Both Senexin A and Senexin B produced concentration-dependent growth inhibition in all three cell lines.

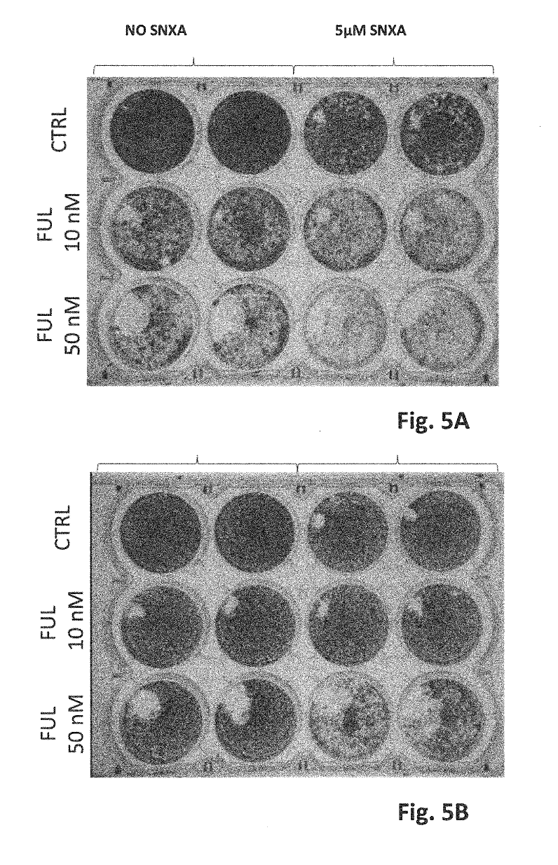

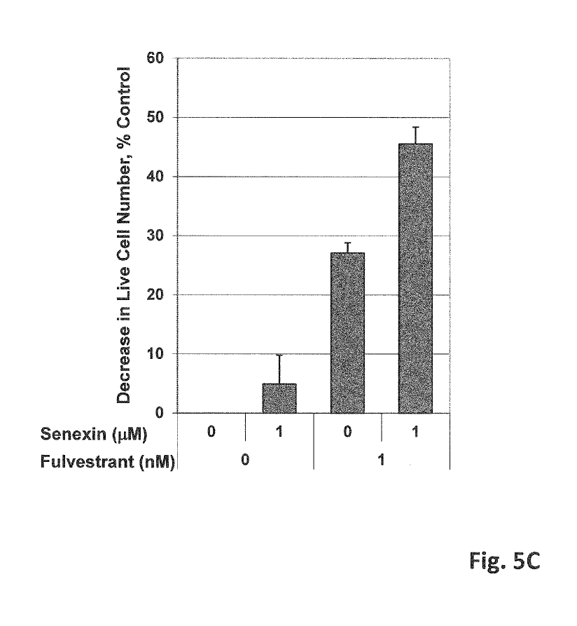

Senexin A showed additive effects with a SEM (tamoxifen) only in the estrogen-dependent lines but not in the estrogen-independent cell lines (Table 1). In contrast, Senexin A (5 μM) showed an additive effect with fulvestrant both in estrogen-dependent lines and in estrogen-independent cell lines (Table 1). The potentiation of fulvestrant effects in MCF7-1pE and MCF7-Veh was especially apparent upon longer (11-day) drug treatment (FIG. 5A and FIG. 5B, showing cells in 12-well plates fixed with methanol and acetic acid and stained with crystal violet). The interaction between Senexin A and fulvestrant in MCF7 cells was investigated in the estrogen (E2) mitogenic stimulation assay, carried out as described in example 1, with the cell number measured by flow cytometry 4 days after E2 addition. Here, E2 was added to estrogen-depleted MCF7 cells in the presence or absence of 1 nM fulvestrant, a selective downregulator of ER, with or without 1 μM Senexin A. As shown in FIG. 5C, 1 nM fulvestrant alone decreased the cell number by 27%, 1 μM Senexin A alone by 5%, and a combination of fulvestrant and Senexin A by 46%, demonstrating a synergistic effect. These results indicate that CDK8/19 inhibitors can be advantageously combined with antiestrogen therapies, such as SERDs (including fulvestrant) and agents that decrease estrogen production (aromatase inhibitors).

### Example 3

### CDK8/19 Inhibition has a Synergistic Effect with HER2/Neu Inhibition in ER+HER2+ Breast Cancer

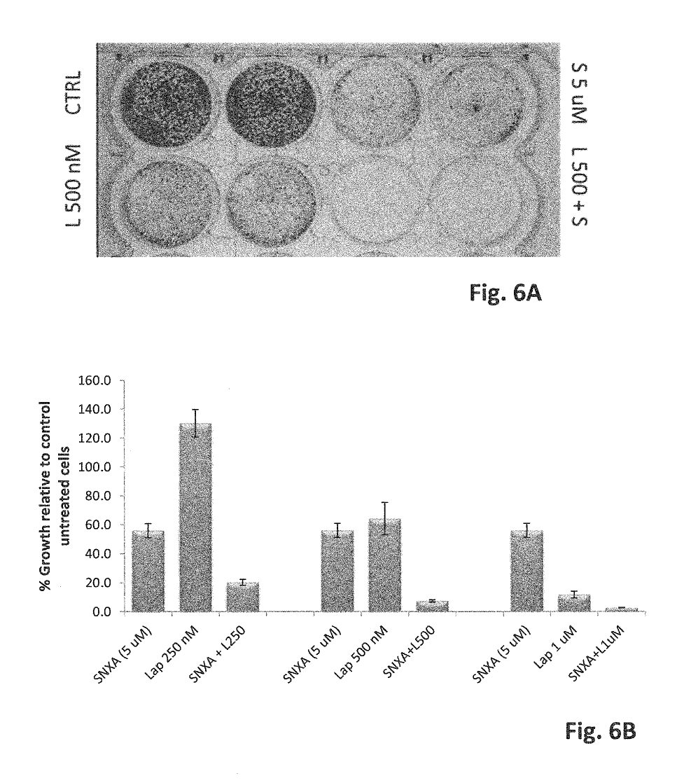

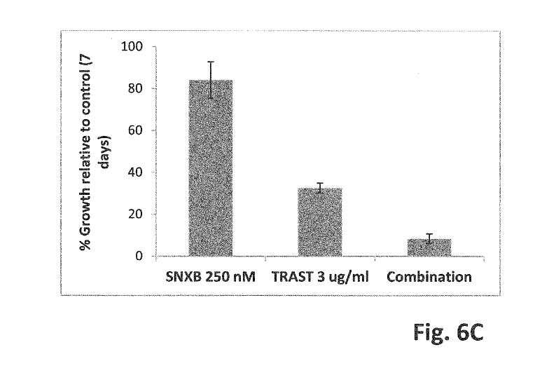

It is known that HER2/Neu overexpression or gene amplification contributes to de novo and acquired resistance to endocrine therapies and that resistance to HER2-targeting agents can be conferred by the upregulation of ER (Wang et al., 2011 and references therein). Since we have found that CDK8/19 inhibition inhibits ER-mediated mitogenic signaling, we hypothesized that CDK8/19 inhibitors could have a synergistic effect with HER2-targeting drugs in ER+HER2+ breast cancer cells. In the experiment in FIG. 6, we have investigated the interaction between Senexin A and lapatinib, a small-molecule inhibitor of HER2 and EGFR, in ER+HER2+BT474 cell line. FIG. 6a shows long-term effects of Senexin A (5 μM) alone and in combinations with lapatinib (LAP) (500 nM) on BT474 cells. Cells were treated for 14 days, then fixed with methanol and acetic acid and stained with crystal violet. FIG. 6b shows relative cell numbers (determined using a Coulter counter) of BT474 cells treated with 5 μM Senexin A in combination with three different concentrations of lapatinib for 15 days. These experiments show a synergistic interaction of the CDK8/19 inhibitor with lapatinib.

While lapatinib acts on both HER2/Neu and EGFR, the widely used drug trastuzumab is a HER2/Neu-specific humanized monoclonal antibody. We have tested Senexin B for the interaction with a trastuzumab biosimilar produced by Biocad (Strelna, Russia) in BT474 cells. In the experiment shown in FIG. 6c, BT474 cells were untreated or treated with Senexin B (250 nM), trastuzumab biosimilar (3 μg/ml) or a combination of both, on days 1, 3 and 5, and the cell numbers were counted (using Coulter counter) on day 7. As shown in FIG. 6c, these selective CDK8/19 and HER2 inhibitors show a synergistic effect.

### Example 4

### CDK8/19 Inhibition Blocks ER-Mediated Induction of Genes Implicated in Estrogen Mitogenic Effect

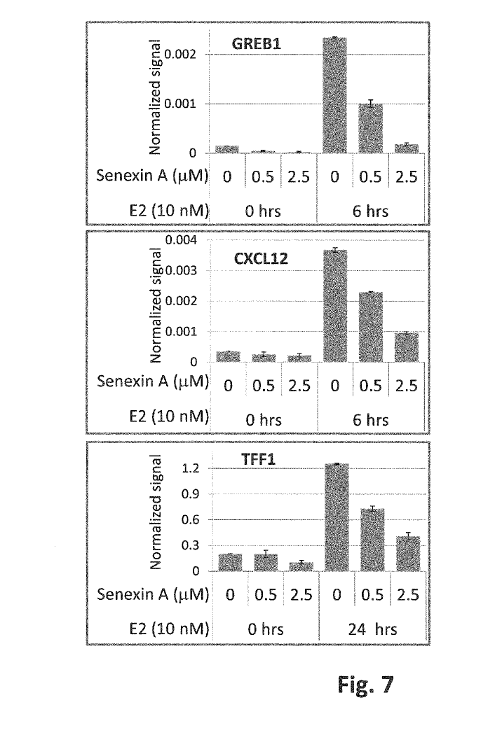

Rae et al., (2005) used microarray profiling to analyze the effects of E2 and ER antagonists on gene expression in three ER+BrCa cell lines, MCF7, T47D and BT-474. Only three genes were found to be significantly induced by E2 and inhibited by ER inhibitors in all three cell lines: GREB1, CXCL12 (a.k.a. SDF-1) and TFF1 (a.k.a. PS2). Of these three genes, TFF1 has not been implicated in cell growth, but GREB1 was demonstrated in the same study to be a mediator of the mitogenic effect of E2 (Rae et al., 2005). The cytokine CXCL12 was also shown to mediate the mitogenic effect of E2 (Sauve et al., 2009) and, together with its receptor CXCR4, to be a key determinant of metastasis in cancers of the breast and other organs (Muller et al., 2001; Rhodes et al., 2011). FIG. 7 shows QPCR analysis of GREB1, CXCL12 and TFF1 expression in estrogen-depleted MCF7 cells, treated with two concentrations of Senexin A with or without E2 addition. All three genes were strongly induced by E2 and inhibited by Senexin A. This result suggests that CDK8 inhibition prevents estrogen stimulation of BrCa cell growth by inhibiting ER-mediated induction of genes implicated in the mitogenic effect of estrogen. The effect of Senexin A on CXCL12, a known mediator of metastasis, is of particular interest as a possible cause of the association of CDK8 expression with shorter RFS.

### Example 5

### The Effect of CDK8 on ER-Inducible Gene Expression is Mediated Through Inhibition of the Transcriptional Effect of ER

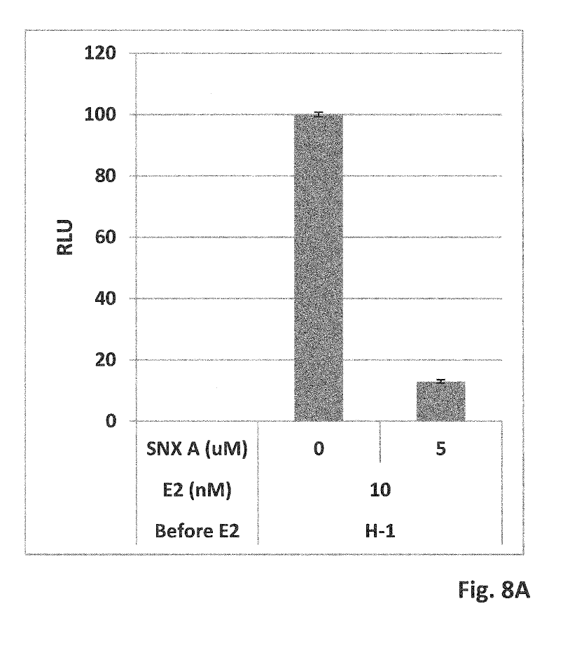

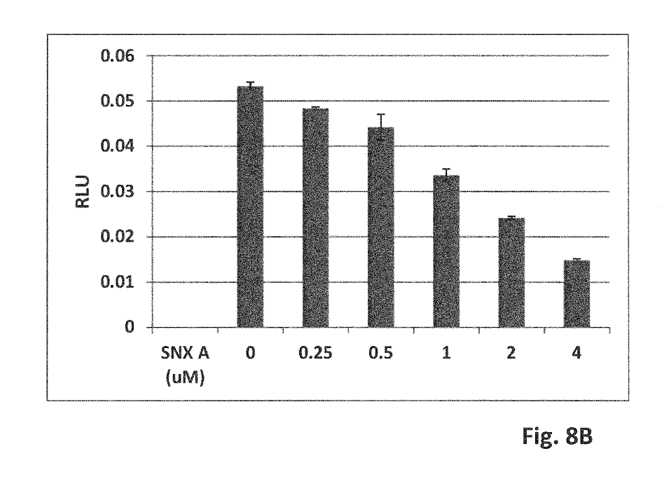

To determine whether the effect of CDK8 on ER-inducible gene expression is mediated through inhibition of the transcriptional effect of ER, which is exerted through ER binding to the ERE promoter element, a T47D-based reporter cell line was used. The cell line T47D-ER/Luc (obtained from Signosis) expresses firefly luciferase from the ER-dependent ERE-containing promoter. The reporter cell line was estrogen-depleted, then Senexin A was added at different concentrations, and one hour later 10 nM E2 was added. 18 hrs later, cells were counted and luciferase activity was measured. Two independent experiments in FIG. 8a and FIG. 8b show that transcription from ER-dependent promoter was strongly inhibited by Senexin A, indicating that CDK8/19 potentiates ER activity.

The results above indicate that CDK8/19 inhibitors, such as Senexin A or Senexin B (see U.S. patent application Ser. No. 13/757,682) should be useful in the treatment of ER+ breast cancers, either alone or in combination with antiestrogen therapies (such as tamoxifen, fulvestrant or aromatase inhibitors). Furthermore, since some of the breast cancers develop resistance to antiestrogen therapy through increased ER activity, allowing them to grow in the presence of greatly reduced estrogen levels, such resistant and difficult to treat cancers are likely to be especially susceptible to CDK8/19 inhibitors. Furthermore, CDK8/19 inhibitors can be advantageously combined with HER2 inhibitors (such as trastuzumab or lapatinib) in the treatment of ER+HER2+ breast cancers.

### REFERENCES

Adler, A. S., McCleland, M. L., Truong, T., Lau, S., Modrusan, Z., Soukup, T. M., Roose-Girma, M., Blackwood, E. M., and Firestein, R. (2012). CDK8 maintains tumor de-differentiation and embryonic stem cell pluripotency. Cancer Res.
Belakavadi, M. and Fondell, J. D. (2010). Cyclin-dependent kinase 8 positively cooperates with Mediator to promote thyroid hormone receptor-dependent transcriptional activation. Mol. Cell Biol. 30, 2437-2448.
Catania, C., Ascione, G., Adamoli, L., De, P. T., Medici, M., Franceschelli, L., Verri, E., Magni, E., Sanna, G., Torrisi, R., Goldhirsch, A., and Nole, F. (2007). Fulvestrant in heavily pre-treated patients with advanced breast cancer: results from a single compassionate use programme centre. Breast Cancer Res. Treat. 106, 97-103.
Firestein, R., Bass, A. J., Kim, S. Y., Dunn, I. F., Silver, S. J., Guney, I., Freed, E., Ligon, A. H., Vena, N., Ogino, S., Chheda, M. G., Tamayo, P., Finn, S., Shrestha, Y., Boehm, J. S., Jain, S., Bojarski, E., Mermel, C., Barretina, J., Chan, J. A., Baselga, J., Tabemero, J., Root, D. E., Fuchs, C. S., Loda, M., Shivdasani, R. A., Meyerson, M., and Hahn, W. C. (2008). CDK8 is a colorectal cancer oncogene that regulates beta-catenin activity. Nature 455, 547-551.
Firestein, R. and Hahn, W. C. (2009). Revving the Throttle on an oncogene: CDK8 takes the driver seat. Cancer Res 69, 7899-7901.
Firestein, R., Shima, K., Nosho, K., Irahara, N., Baba, Y., Bojarski, E., Giovannucci, E. L., Hahn, W. C., Fuchs, C. S., and Ogino, S. (2010). CDK8 expression in 470 colorectal cancers in relation to beta-catenin activation, other molecular alterations and patient survival. Int. J. Cancer 126, 2863-2873.
Galbraith, M. D., Donner, A. J., and Espinosa, J. M. (2010). CDK8: a positive regulator of transcription. Transcription. 1, 4-12.
Kapoor, A., Goldberg, M. S., Cumberland, L. K., Ratnakumar, K., Segura, M. F., Emanuel, P. O., Menendez, S., Vardabasso, C., Leroy, G., Vidal, C. I., Polsky, D., Osman, I., Garcia, B. A., Hernando, E., and Bernstein, E. (2010). The histone variant macroH2A suppresses melanoma progression through regulation of CDK8. Nature 468, 1105-1109.
McDonnell, D. P. and Wardell, S. E. (2010). The molecular mechanisms underlying the pharmacological actions of ER modulators: implications for new drug discovery in breast cancer. Curr. Opin. Pharmacol. 10, 620-628.
Muller, A., Homey, B., Soto, H., Ge, N., Catron, D., Buchanan, M. E., McClanahan, T., Murphy, E., Yuan, W., Wagner, S. N., Barrera, J. L., Mohar, A., Verastegui, E., and Zlotnik, A. (2001). Involvement of chemokine receptors in breast cancer metastasis. Nature 410, 50-56.
Porter, D. C., Farmaki, E., Altilia, S., Schools, G. P., West, D. K., Chen, M., Chang, B. D., Puzyrev, A. T., Lim, C. U., Rokow-Kittell, R., Friedhoff, L. T., Papavassiliou, A. G., Kalurupalle, S., Hurteau, G., Shi, J., Baran, P. S., Gyorffy, B., Wentland, M. P., Broude, E. V., Kiaris, H., and Roninson, I. B. (2012). Cyclin-dependent kinase 8 mediates chemotherapy-induced tumor-promoting paracrine activities. Proc. Natl. Acad. Sci. U. S. A 109, 13799-13804.
Rae, J. M., Johnson, M. D., Scheys, J. O., Cordero, K. E., Larios, J. M., and Lippman, M. E. (2005). GREB 1 is a critical regulator of hormone dependent breast cancer growth. Breast Cancer Res. Treat. 92, 141-149.
Rhodes, L. V., Short, S. P., Neel, N. F., Salvo, V. A., Zhu, Y., Elliott, S., Wei, Y., Yu, D., Sun, M., Muir, S. E., Fonseca, J. P., Bratton, M. R., Segar, C., Tilghman, S. L., Sobolik-Delmaire, T., Horton, L. W., Zaja-Milatovic, S., Collins-Burow, B. M., Wadsworth, S., Beckman, B. S., Wood, C. E., Fuqua, S. A., Nephew, K. P., Dent, P., Worthylake, R. A., Curiel, T. J., Hung, M. C., Richmond, A., and Burow, M. E. (2011). Cytokine receptor CXCR4 mediates estrogen-independent tumorigenesis, metastasis, and resistance to endocrine therapy in human breast cancer. Cancer Res. 71, 603-613.
Sauve, K., Lepage, J., Sanchez, M., Heveker, N., and Tremblay, A. (2009). Positive feedback activation of estrogen receptors by the CXCL12-CXCR4 pathway. Cancer Res. 69, 5793-5800.
Sikora, M. J., Strumba, V., Lippman, M. E., Johnson, M. D., and Rae, J. M. (2012). Mechanisms of estrogen-independent breast cancer growth driven by low estrogen concentrations are unique versus complete estrogen deprivation. Breast Cancer Res. Treat. 134, 1027-1039.
Sweeney, E. E., McDaniel, R. E., Maximov, P. Y., Fan, P., and Jordan, V. C. (2012). Models and Mechanisms of Acquired Antihormone Resistance in Breast Cancer: Significant Clinical Progress Despite Limitations. Horm. Mol. Biol. Clin. Investig. 9, 143-163.
Wang Y C, Morrison G, Gillihan R, Guo J, Ward R M, Fu X, Botero M F, Healy N A, Hilsenbeck S G, Phillips G L, Chamness G C, Rimawi M F, Osborne C K, Schiff R (2011). Different mechanisms for resistance to trastuzumab versus lapatinib in HER2-positive breast cancers—role of estrogen receptor and HER2 reactivation. Breast Cancer Research 2011, 13:R121
Westerling, T., Kuuluvainen, E., and Makela, T. P. (2007). Cdk8 is essential for preimplantation mouse development. Mol. Cell Biol. 27, 6177-6182.
Wilson, T. R., Fridlyand, J., Yan, Y., Penuel, E., Burton, L., Chan, E., Peng, J., Lin, E., Wang, Y., Sosman, J., Ribas, A., Li, J., Moffat, J., Sutherlin, D. P., Koeppen, H., Merchant, M., Neve, R., and Settleman, J. (2012). Widespread potential for growth-factor-driven resistance to anticancer kinase inhibitors. Nature 487, 505-509.
Xu, W. and Ji, J. Y. (2011). Dysregulation of CDK8 and Cyclin C in tumorigenesis. J. Genet. Genomics 38, 439-452.

What is claimed is:

1. A method for treating a patient having estrogen receptor positive (ER+) breast cancer comprising administering to the patient an effective amount of a selective inhibitor of CDK8/19.

2. The method according to claim 1, wherein the breast cancer is resistant to antiestrogen therapy.

3. The method according to claim 1, wherein cells of the breast cancer express one or more gene selected from the group consisting of GREB1, CXCL12, and TFF.

4. The method according to claim 2, wherein cells of the breast cancer express one or more gene selected from the group consisting of GREB1, CXCL12, and TFF.

5. The method according to claim 1, further comprising treating the patient with antiestrogen therapy.

6. The method according to claim 5, wherein the antiestrogen therapy comprises administering to the patient an agent selected from a selective estrogen receptor modulator, a selective estrogen receptor downregulator and an aromatase inhibitor.

7. The method according to claim 6, wherein the selective estrogen receptor modulator is selected from tamoxifen, raloxifine and toremifine.

8. The method according to claim 6, wherein the selective estrogen receptor downregulator is fulvestrant.

9. The method according to claim 6, wherein the aromatase inhibitor is selected from anastrozole, exemestane and letrozole.

10. The method according to any of claims 1-9, wherein the selective inhibitor of CDK8/19 has the structural formula I or II: 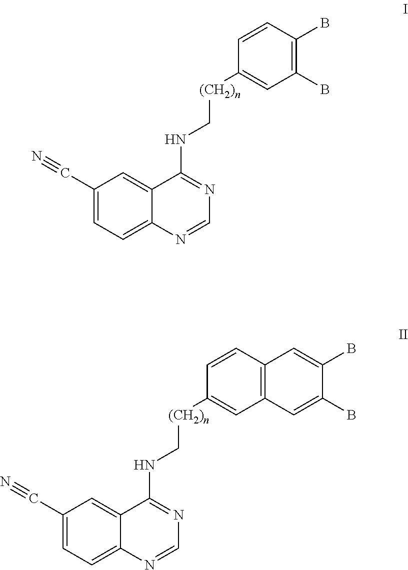
wherein each B is independently hydrogen or 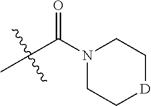
provided that at least one B is hydrogen and not more than one B is hydrogen;
D is selected from —NH, —N-lower alkyl, or O;
and n is 0-2.

11. The method according to claim 10, wherein lower alkyl is methyl.

12. The method according to claim 10, wherein n is 0 or 1.

13. The method according to claim 10, wherein the selective inhibitor of CDK8/19 is selected from the group consisting of SNX2-1-162, SNX2-1-163, SNX2-1-164, SNX2-1-165, SNX2-1-166 and SNX2-1-167.

14. The method according to claim 13, wherein the selective inhibitor of CDK8/19 is SNX2-1-165.

15. The method according to any of claims 1-9, wherein the selective inhibitor of CDK8/19 is selected from the compounds shown in FIG. 1.

16. The method according to claim 10, wherein the selective inhibitor of CDK8/19 is administered orally.

17. The method according to claim 1, wherein the breast cancer is ER+HER2+ and the selective inhibitor of CDK8/19 is administered in combination with a HER2+ inhibitor.

18. The method according to claim 17, wherein the HER2+ inhibitor is selected from lapatinib and trastuzumab.
